(19) United States
(12) Patent Application Publication
(10) Pub. No.: US 2022/0110762 A1
(43) Pub. Date: Apr. 14, 2022

(54) Spinal Prothesis

(71) Applicant: Musgrave Solutions LLC, St. Louis, MO (US)

(72) Inventors: Nicholas J. Musgrave, St. Louis, MO (US); Steven A. Sauer, Upper Arlingtion, OH (US)

(73) Assignee: Musgrave Solutions LLC, St. Louis, MO (US)

(21) Appl. No.: 17/499,535

(22) Filed: Oct. 12, 2021

Related U.S. Application Data
(60) Provisional application No. 63/090,341, filed on Oct. 12, 2020.

(51) Int. Cl. A61F 2/44; A61F 2/30

(52) U.S. Cl. CPC A61F 2/4455; A61F 2/30734; A61F 2002/30069; A61F 2002/30172; A61F 2002/30378

(57) ABSTRACT

A spinal prosthesis is provided, more particularly a spinal prosthesis having component parts capable of assembly during surgery in a spinal environment for providing stability and flexibility to the spine. The component parts include a ball-and-socket combination, a compressible pad, and anchors at each end of the prosthesis to secure the prosthesis in the spine.

### CROSS-REFERENCE TO RELATED APPLICATIONS

This application claims priority to U.S. Pat. App. No. 63/090,341, filed Oct. 12, 2020, entitled “Spinal Prosthesis”, currently pending, which application is incorporated herein by reference.

### FIELD OF INVENTION

The present invention relates generally to prostheses for vertebra and intervertebral discs, and more particularly, but without limitation, to apparatus and methods of making and methods of treating degenerative spine disorders, diseases of the vertebral body, intervertebral disc, or both, traumatic injury, and tumors of the spinal column.

### BACKGROUND

Problems with the spine are one of the leading causes of pain, suffering, and disability in the United States today. Such pain can be either chronic over months or years, or acute and intense. Its etiologies, or causes, are generally multifactorial and the problems and suffering spinal pain causes may be debilitating. Furthermore, the costs to society in terms of quality-of-life issues due to pain and suffering, medical care costs, and loss of economic productivity are significant.

Whether musculoskeletal with its effects limited to nuisance, neoplastic with a dismal prognosis, or neurological with effects ranging to catastrophic paralysis, spinal pain is treated with either nonsurgical methods, including pain management, such as medication, including epidural steroid injections, physical therapy, meditation, yoga, acupuncture, and homeopathic measures, or surgery, or combinations. At present, surgical intervention is generally reserved for those individuals who have failed multiple interventions of medical (nonsurgical) management, or who are developing rapidly progressing neurological symptoms, which, if left untreated, may result in permanent disability. When surgery is indicated, the customary procedure involves removing a portion of diseased or damaged intervertebral disc or bone, and, if the remaining spine is determined to be unstable, installing connecting reinforcing metal plates, screws, and rods to hold the vertebrae together. This procedure is commonly known as “spinal fusion” and is used to treat such conditions as spondylitis, spondylolisthesis, degenerative joint disease, degenerative disc disease, discitis, disc herniation, central cord syndrome, cervical radiculopathy, tumor removal, arthritis, synovial cyst, trauma stabilization, osteomyelitis, epidural hematoma, epidural abscess, spinal cord metastasis, laminectomy, and revision surgery.

### SUMMARY

Prosthetic surgical implants designed to replace diseased or injured vertebral bone and diseased or injured cartilaginous discs are disclosed. Particularly, the prosthesis comprises at least one, and preferably two anchors to allow the prosthesis to be secured in place in the spinal environment, one or more articulating elements, such as ball-and-socket joints or couplings operably connected to the at least one anchor to allow at least some degree of multidirectional movement and rotation. Compressible elements may be operably connected to provide stability and flexibility to the prosthesis in use. In cases where one or more vertebrae are removed, the prosthesis may also include one or more spacers to provide proper sizing in the spinal environment. Alternatively, the prosthesis comprises an least one, and preferably two anchors to allow the prosthesis to be secured in place in the spinal environment, one or more elastic elements, such as springs or biocompatible polymers, operably connected to provide stability to the prostheses in use. In use, the prosthesis provides stability to the affected area of the spine while allowing for the natural flexibility of the native spine. Also disclosed are methods of making and methods of implanting said apparatus.

The present disclosure is related to a spinal prosthesis comprising a first anchor, the first anchor including an annular groove; a ball component of a ball-and-socket combination, the ball component including a ball attached to a base and including an annular skirt adapted to mate with the annular groove of the first anchor; and a second anchor, the second anchor including a socket adapted to receive the ball. Interposed between the second anchor and the base of the ball component is a compressible pad, the compressible pad including a hole adapted to accept the ball.

The present disclosure is further related to a spinal prothesis comprising a first anchor, the first anchor including a base adapted to be secured to a vertebra and the ball of a ball-and-socket combination; and a second anchor, the second anchor including a base adapted to be secured to a vertebra and the socket of a ball-and-socket combination, the socket adapted to mate with the ball.

The present disclosure is further related to a spinal prosthesis comprising a vertebral spacer having a first surface and a second surface, first and second spheroid domes attached, respectively, to each of the first and the second vertebral spacer surfaces; first and second anchors, each of the first and the second anchors spaced apart, respectively, from the first and the second vertebral spacer surfaces, each of the first and the second anchors adapted to be secured to a vertebra; and a first and a second spheroidal cup, each of the first and second spheroidal cups operatively attached, respectively, to each of the first and the second anchor by a first and a second spring, each of the first and the second spheroidal cup oriented to and adapted to mate, respectively, with each of the first and the second spheroidal domes. Each of the first spring and the second spring adapted to be elastic in at least compression, but may also be elastic in tension and elastic in torsion.

The present disclosure is further related to a spinal prosthesis comprising a vertebral spacer having a first surface and a second surface; a first spherical dome operatively attached to the vertebral spacer first surface by a first elastic element, and a second spherical dome operatively attached to the vertebral spacer second surface by a second elastic element. The spinal prosthesis further comprises a first anchor adapted to be secured to a vertebra, the first anchor spaced apart from the vertebral spacer first surface, and a second anchor adapted to be secured to a vertebra, the second anchor spaced apart from the vertebral spacer second surface; a first spheroidal cup operatively attached to the first anchor, and a second spheroidal cup operatively attached to the second anchor. The first spheroidal cup oriented to and adapted to mate with the first spheroidal dome and the second spheroidal cup oriented to and adapted to mate with the second spheroidal dome.

The present disclosure is further related to a spinal prosthesis comprising a vertebral spacer having a first surface and a second surface; a first spheroidal dome operatively attached to the vertebral spacer first surface and a second spheroidal dome operatively attached to the vertebral spacer second surface; a first anchor adapted to be secured to a vertebra, the first anchor spaced apart from the vertebral spacer first surface; a second anchor adapted to be secured to a vertebra, the second anchor spaced apart from the vertebral spacer second surface; a first spheroidal cup, the first spheroidal cup operatively attached to the first anchor by a first elastic element, and a second spheroidal cup, the second spheroidal cup operatively attached to the second anchor by a second elastic element, the first spheroidal cup oriented to and adapted to mate with the first spheroidal dome, and the second spherical cup oriented to and adapted to mate with the second spheroidal dome. Each of the first elastic element and the second elastic element adapted to be elastic in at least compression.

The present disclosure is further related to a spinal prosthesis comprising a vertebral spacer having a first surface and a second surface; a first anchor adapted to be secured to a vertebra, the first anchor spaced apart from the vertebral spacer first surface, the first anchor operatively attached to the vertebral spacer first surface by a first elastic element; a second anchor adapted to be secured to a vertebra, the second anchor spaced apart from the vertebral spacer second surface, the second anchor operatively attached to the vertebral spacer second surface by a second elastic element. Each of the first elastic element and the second elastic element adapted to be elastic in compression, tension, and torsion.

The present disclosure is further related to a spinal prosthesis having component parts capable of being assembled in vivo in a spinal environment for replacement of vertebra and adjacent discs to provide stability while allowing for the natural flexibility of the native spine. The spinal prosthesis comprises a first anchor and a second anchor, each anchor adapted to allow the prosthesis to be secured in place in the spinal environment. Operatively connected to the first anchor is a first cup and operatively connected to the second anchor is a second cup. The spinal prosthesis further comprises a spacer, the spacer having first and second opposing sides. Operatively connected to the first side with a biocompatible spring or elastic element is a first dome and operatively connected to the second side with a spring is a second dome. The first cup is capable of being oriented and adapted to mate with the first dome and the second cup is capable of being oriented to mate with the second dome.

The present disclosure is further related to a spinal prosthesis comprising prostheses described herein above and further comprising flexible sleeves enclosing the elastic elements.

The present disclosure is further related to a method of making a spinal prosthesis comprising the steps of fashioning first and second anchors adapted to attach to a vertebra; forming a ball having a base, the base adapted to mate with the first anchor; mating and securing the base to the first anchor; forming a compressible pad comprising an aperture and installing the pad over the ball through the aperture; and mating the second anchor to the ball.

The present disclosure is further related to a method of implanting a spinal prosthesis comprising the steps of exposing the subject vertebrae (e.g., C4-C5-C6) and removing the body (corpus vertebrae) of the subject vertebra (e.g., C5) and optionally adjacent discs; shaping and preparing the anchoring vertebra (e.g., C4 and C6) to accept the anchors; attaching an anchor having an attached ball to the lower vertebra (e.g., C6); attaching a compressible pad over the ball through an aperture in the pad; and mating a second anchor having a socket to the ball; and attaching the second anchor to the upper vertebra.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the embodiment in the view.

FIG. 1 is a perspective view of a spinal prosthesis according to an embodiment of the present invention.

FIG. 2 is a cross-section view of the spinal prosthesis of FIG. 1 along the plane 2-2-2 of FIG. 1 showing various components.

FIG. 3 is a further perspective view of a spinal prosthesis according to an embodiment of the present invention.

FIG. 4 is a cross-section view of the spinal prosthesis of FIG. 3 along the plane 4-4-4 of FIG. 3 showing various components.

FIGS. 5 and 6 are exploded perspective views of a spinal prosthesis according to an embodiment of the present invention.

FIG. 7 is an exploded cross-section perspective view of a spinal prosthesis according to an embodiment of the present invention.

FIGS. 8 and 9 are exploded cross-section perspective views showing a partial assembly of a spinal prosthesis according to an embodiment of the present invention.

FIGS. 10 and 11 are perspective views of a spinal prosthesis according to an embodiment of the present invention showing partial rotation of the prosthesis.

FIG. 12 is an elevation view of a spinal prosthesis according to an embodiment of the present invention showing partial bending of the prosthesis.

FIGS. 13 and 14 are exploded perspective views of a spinal prosthesis according to a further embodiment of the present invention.

FIGS. 15 and 16 are exploded perspective views of a spinal prosthesis according to a further embodiment of the present invention.

FIG. 17 is an exploded perspective view of a spinal prosthesis according to a further embodiment of the present invention.

FIG. 18 is a further exploded perspective view of the spinal prosthesis of FIG. 17 according to a further embodiment of the present invention.

FIG. 19 is a perspective view of a spinal prosthesis according to a further embodiment of the present invention.

FIGS. 20 and 21 are exploded perspective views of a spinal prosthesis according to a further embodiment of the present invention.

FIG. 22 is a partial cutaway elevation view of a spinal prosthesis according to a further embodiment showing a flexible sleeve enclosing a portion of the prothesis.

FIG. 23 is an elevation view of a further embodiment of the spinal prosthesis according to the present invention.

FIG. 24 is an elevation view of a further embodiment of a spinal prosthesis according to the present invention.

FIG. 25 is a perspective view of C4-C6 vertebrae in place.

FIG. 26 is an exploded perspective view of C4-C6 vertebrae prepared for implantation of a spinal prothesis according to the present invention.

FIG. 27 is an exploded perspective view of C4-C6 vertebrae with a spinal prosthesis according to the present invention implanted.

FIG. 28 is a perspective view of C4-C6 vertebrae with a spinal prothesis implanted.

### DETAILED DESCRIPTION

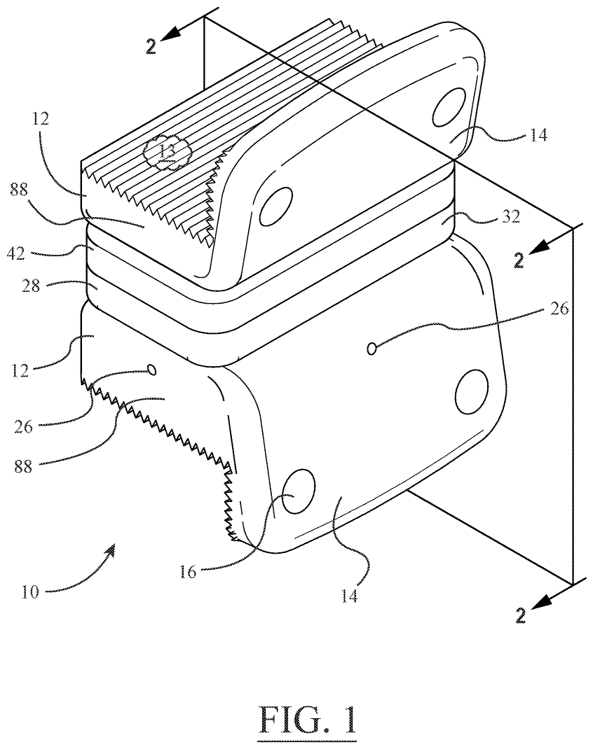

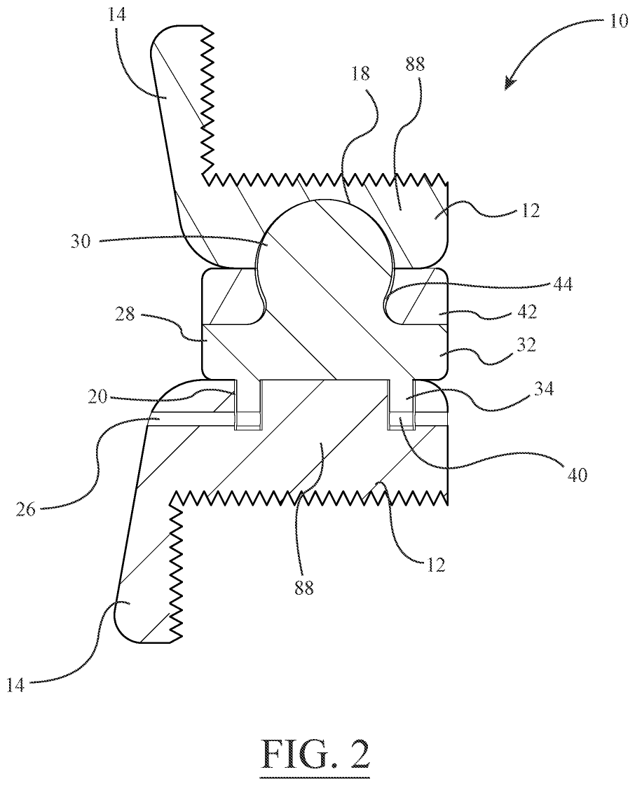

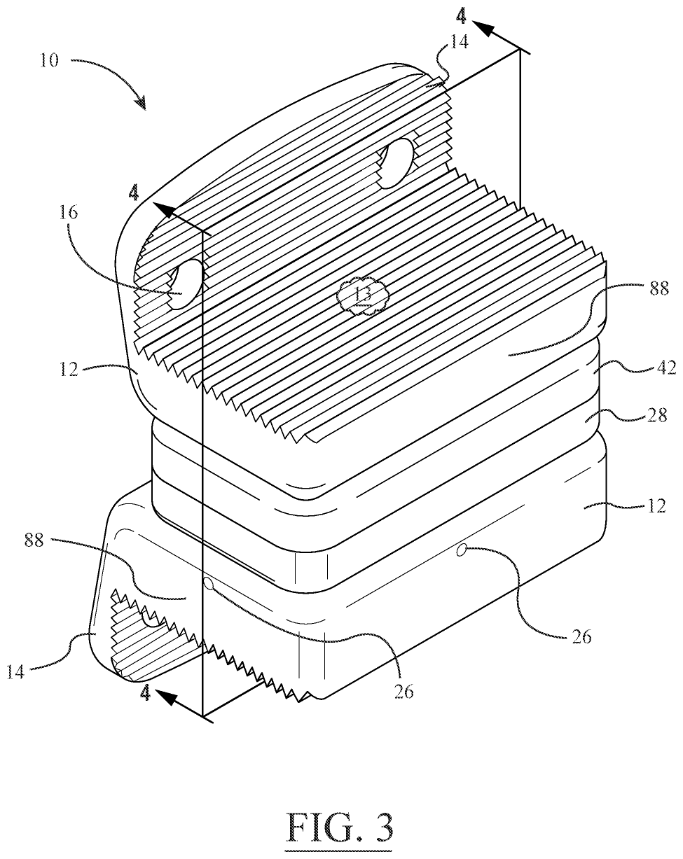

FIG. 1 shows an embodiment of a spinal prosthesis 10. At the top and bottom are anchors 12, each with a body 88 and a leg 14, the leg 14 with anchor leg connector holes 16 for securing to a vertebra. As shown, the anchors 12 may be angular to attach to two surfaces. The anchors 12 include surfaces 13 that are fashioned or otherwise treated to encourage bone growth into the anchors 12. Situated between the anchors 12 are a compressible pad 42 atop a ball component 28, the ball component 28 comprising a base 32 and a ball of a ball-and-socket combination 30. Next, FIG. 2 is a cross-section view taken along the plane 2-2-2 of FIG. 1. The top anchor 12 comprises a socket 18 of a ball-and-socket combination. As shown in FIG. 2, the ball 30 of the ball component 28 movably mates with the socket 18 of the top anchor 12. The ball component base 32 further comprises a skirt 34 which is adopted to mate with a skirt groove 20 of the bottom anchor 12. Next, the bottom anchor 12 further comprises at least one anchor duct 26 (also in FIGS. 1 and 3) and at least one skirt duct 40 for use in assembling the prosthesis 10 (described herein below). Finally, the compressible pad 42 is installed over the ball 30 where it is further supported by the ball component base 32. The pad hole 44 may be sized to provide a stretch fit over the ball 30 so the pad 42 fits snuggly on the ball component 28.

While these figures show an embodiment with an initially separate bottom anchor 12 and ball component 32 which are later secured together, it is to be understood that the bottom anchor 12 and the ball component 32 may be formed together, thereby skipping the step of securing the ball component 32 to the bottom anchor 12.

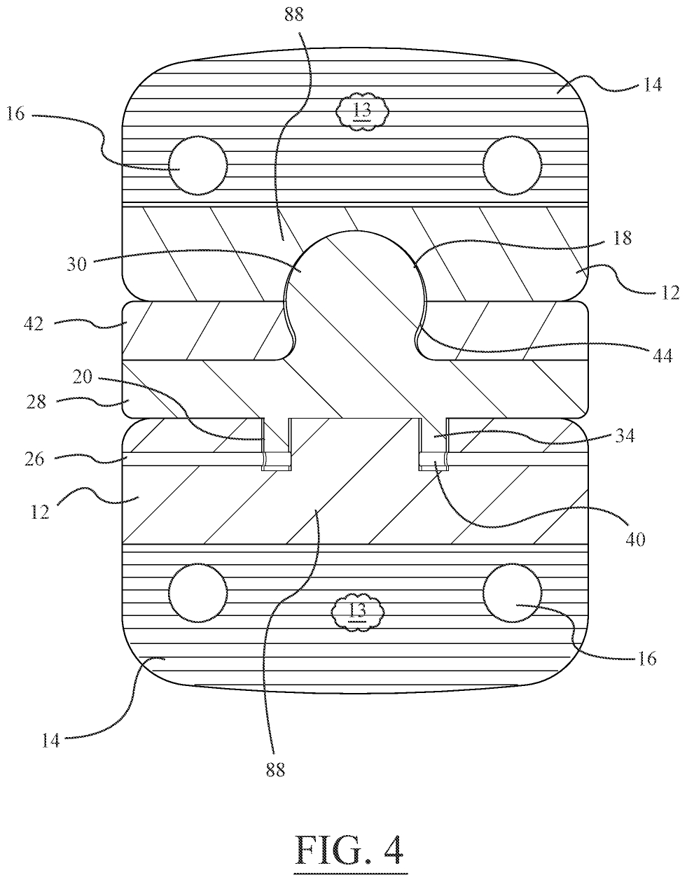

FIG. 3 shows an embodiment of a spinal prosthesis 10. Visible are the anchors 12, anchor ducts 26, ball component 28, and pad 42. FIG. 4 is a cross-section along the plane 4-4-4 of FIG. 3. Features shown in FIG. 4 are the analogous features of FIG. 2.

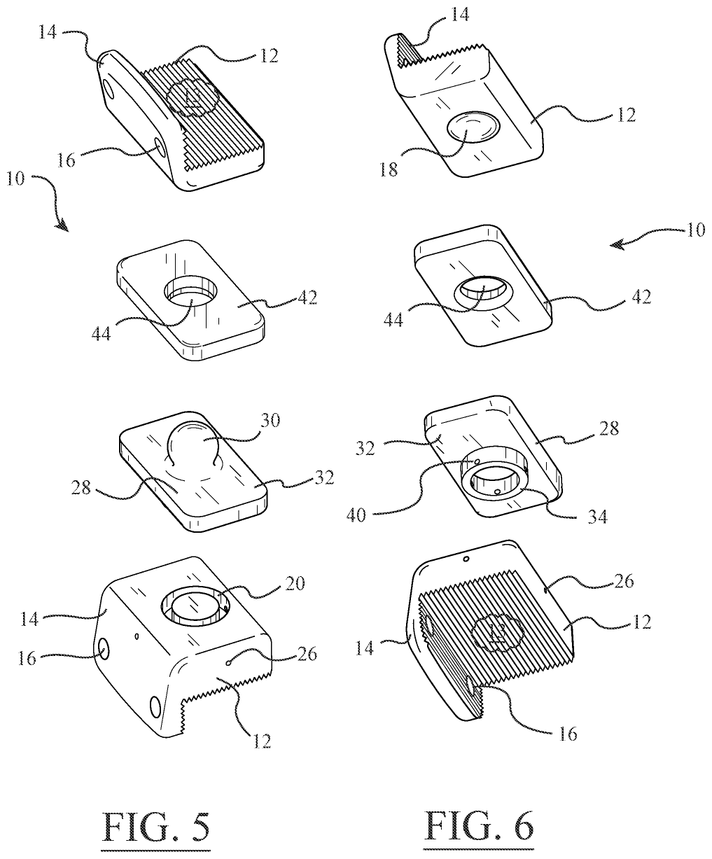

Turning now to FIGS. 5 and 6, shown are exploded perspective views from above and below of a spinal prosthesis 10. More clearly visible in FIG. 5 is the skirt groove 20 of the bottom anchor 12 along with one of its associated anchor ducts 26. Likewise, in FIG. 6, the ball component skirt 34 together with its associated ball component skirt ducts 40 are seen.

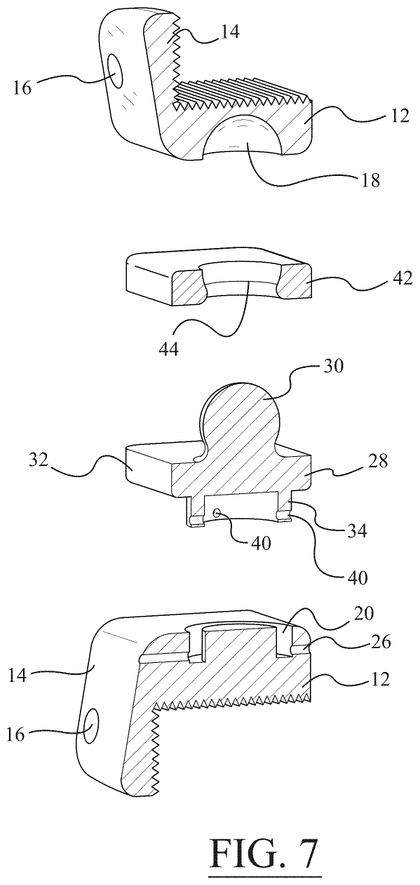

FIG. 7 is an exploded cross-section of a spinal prosthesis 10. Shown are the ball component skirt 34, the anchor ball component skirt groove 20, the anchor ducts 26, and the ball component skirt ducts 40.

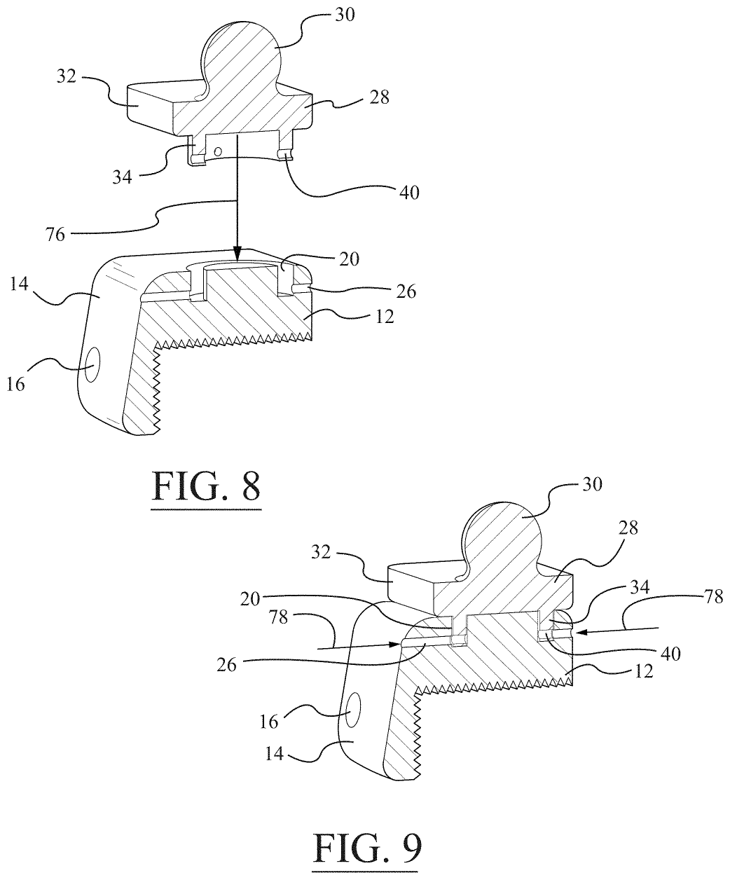

FIGS. 8 and 9 demonstrate the assembly process of the ball component 28 with the bottom anchor 12. The anchor 12 may be constructed from surgical steel designed for biomedical use as implanted in a patient. Such parts may be fashioned using a Computer Numerically Controlled (CNC) to mill the part down from a solid block of metal. In addition, details such as the textured surface pattern 13 as well as the anchor ducts 26 may be achieved with a CNC process.

The ball component 28 that will be mated to the anchor 12 (arrow 76) may be formed of medical grade silicone (Class V and VI) using injection molding. Silicones having a Shore Hardness of Shore A between 40-80 would provide the necessary balance between flexibility and rigidity. (The Shore Hardness Scale consists of three overlapping scales (Shore 00, Shore A, and Shore D. The Shore A range is between 0-100.)

FIG. 9 shows the ball component 28 mated to the anchor 12. The step shown in FIG. 9 with arrows 78 represents two of the four injections of molten silicone under high heat and pressure into the void between the skirt 34 and the skirt groove 20. The injected silicone is then allowed to cool and harden to fuse to the skirt 34 and the anchor groove 20. As shown in FIGS. 8 and 9, the ball component skirt 34 mates with the anchor skirt groove 20.

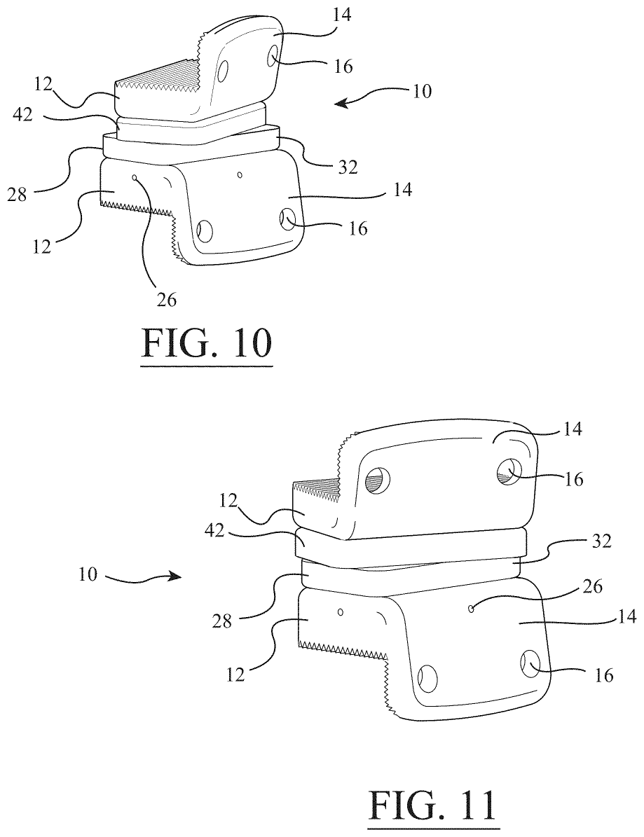

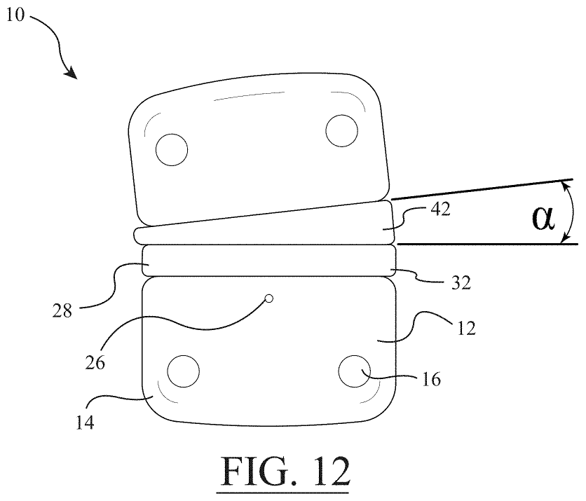

FIGS. 10 and 11 illustrate how the spinal prosthesis 10 can rotate about an axis through the ball-and-socket combination. FIG. 12 illustrates how the spinal prosthesis 10 may tilt with the pad 42 to an angle α, generally about 10 degrees.

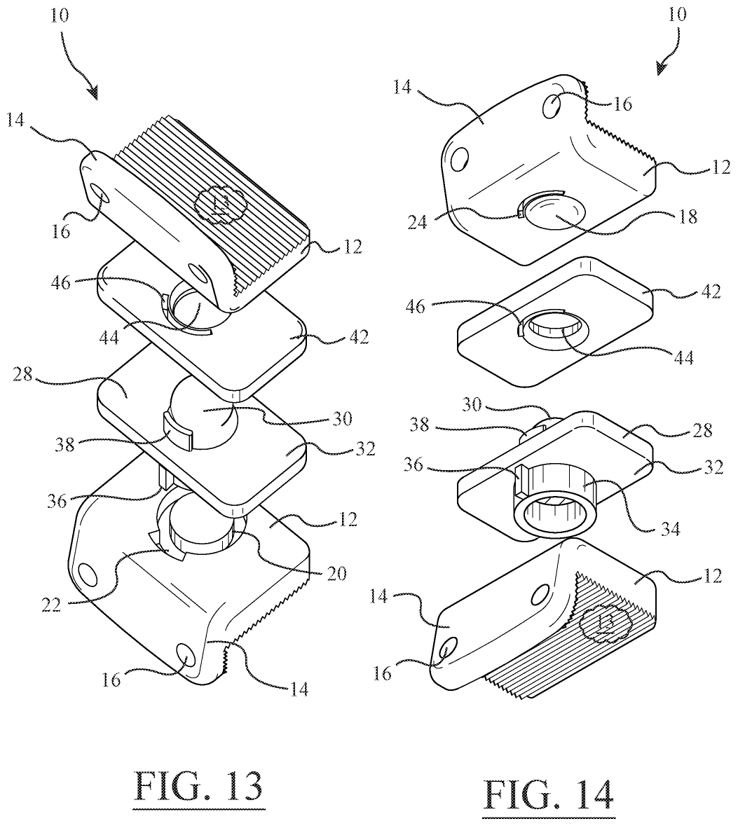

FIGS. 13 and 14 illustrate two stop mechanisms to limit the rotational motion of the spinal prosthesis 10. It is to be understood that the spinal prosthesis 10 may have no stop mechanism, one stop mechanism, or multiple stop mechanisms. Looking at FIGS. 13 and 14, the ball component skirt 34 further includes a tab stop 36 which is adapted to ride in an anchor ball component tab stop notch 22. Another rotational motion limiting mechanism is defined by a ball component arch stop 38 and an anchor ball component arch stop groove 24. To accommodate this latter arrangement, a pad cut 46 is included in the pad 42.

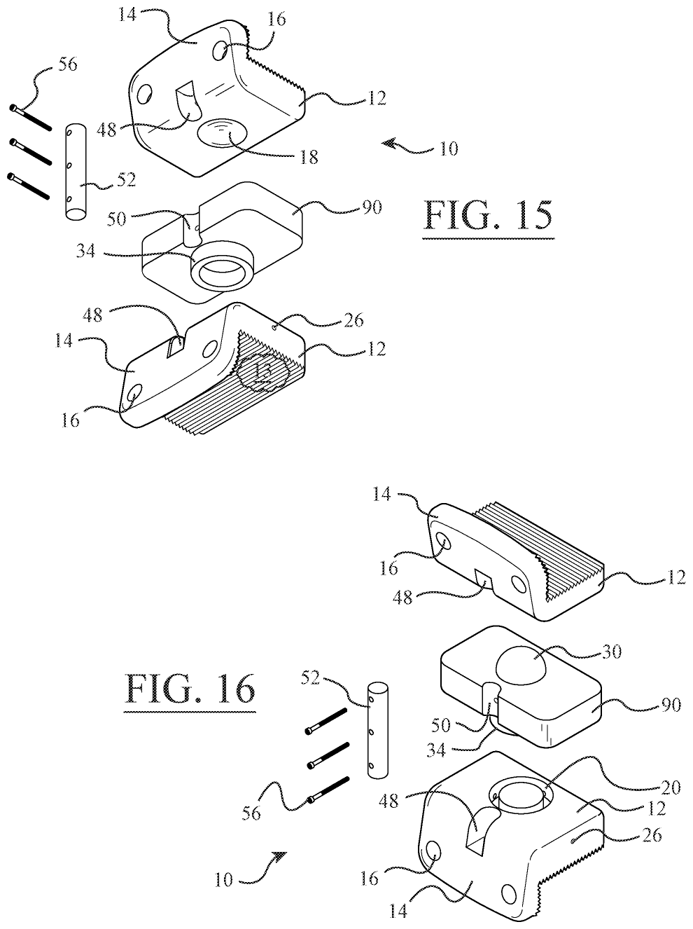

Turning now to FIGS. 15 and 16, a spinal prosthesis 10 comprises top and bottom anchors 12 with a rigid spacer 88 therebetween. The spacer 88 also includes a ball portion 30 to mate with a socket portion 18 of the top anchor 12. Further included is a connector 52 to secure the two anchors 12 and the rigid spacer 88. In the event a patient cannot tolerate the more flexible spinal prosthesis described herein, this device can be converted to a spinal fusion. In addition, if the pad 42 deteriorates and must be replaced, such a device can facilitate the changeout.

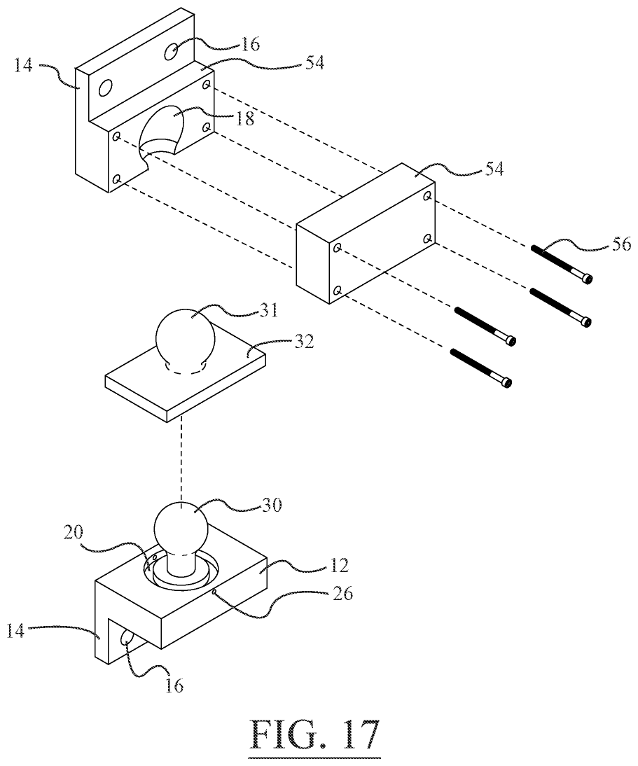

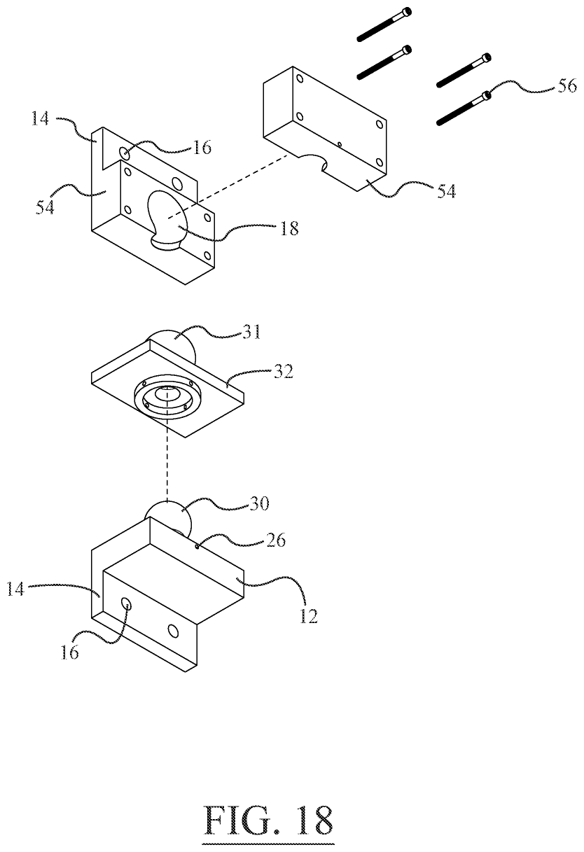

FIGS. 17 and 18 illustrate a further spinal prosthesis 10. As shown, a bottom anchor 12 comprises a rigid ball 30 and a skirt groove 20; a base 32 with a flexible sleeve 31 which flexible sleeve is adapted to accept the rigid ball 30 of the bottom anchor 12. Finally, the multi-piece socket block 54 (shown as two-pieces) is adapted to fit over the rigid ball 30 covered by the flexible sleeve 31 and secured by screws 56 and becomes the top anchor 12. This prosthesis 10 can be used when motion at the joint is desired, but intended to be more limited than other embodiments. It may be useful in younger patients with more mobile spines where too much motion could be detrimental to the spinal cord. Also, it can be used when patients suffer from distraction injury, or where there is significant concern for distractive forces that could potentially pull the ball 30 from the socket 80.

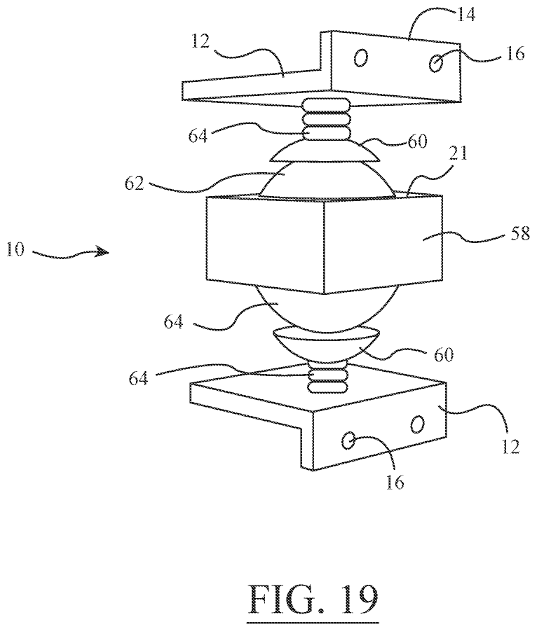

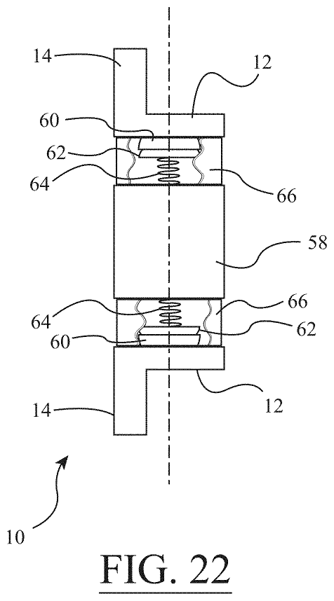

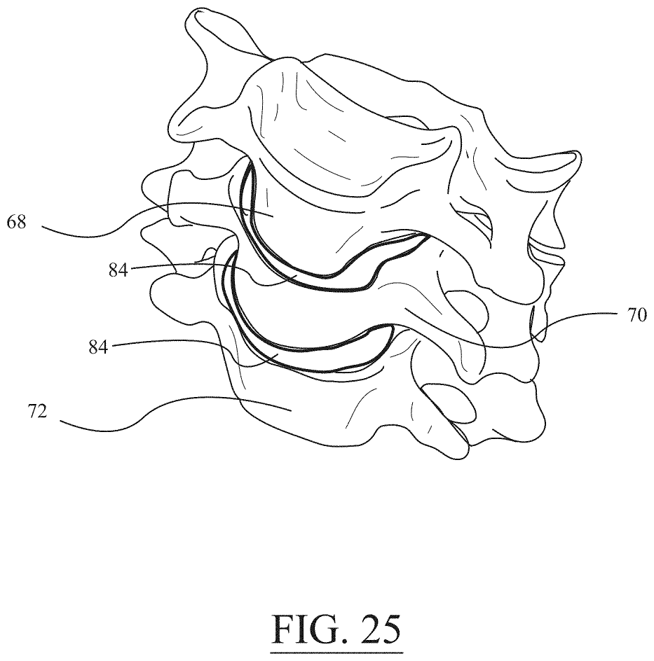

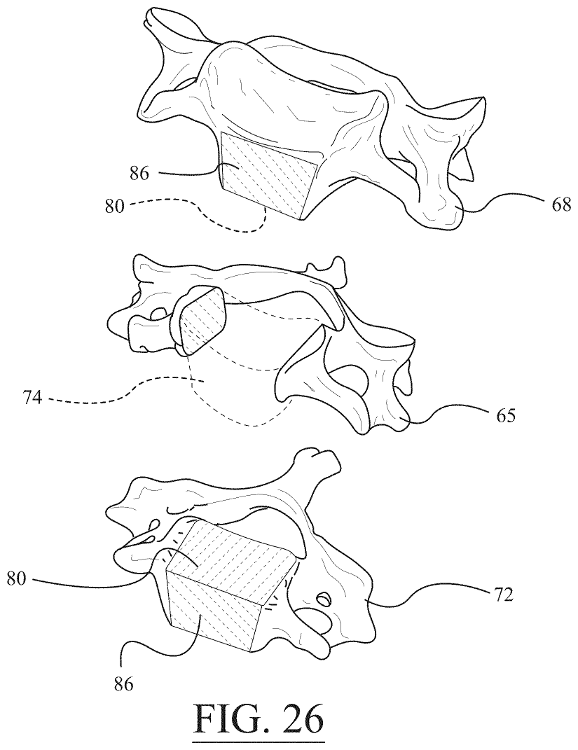

FIG. 19 illustrates a further spinal prosthesis 10. A vertebral spacer 58 may be generally central in an axis to the prosthesis 10 and may be sized to cooperate with other elements of the prosthesis 10 to suitably occupy the gap created when a portion of a vertebra 70 (e.g., C5) and its adjacent discs 84 (FIGS. 25 and 26) are removed from the spine. While the vertebral spacer 58 is shown as a rectangular cuboid with right edges and right vertices, in practice the spacer 58 may have rounded edges and vertices or other shapes. Secured to opposing surfaces of the spacer 58 are spheroidal domes 62. The spinal prosthesis 10 further comprises first and second anchors 12, each first and second anchor 12 adapted to be secured to a vertebra 68, 72 (FIGS. 25-28). Operatively attached to each anchor 12 by a first and second elastic element 64 is a spheroidal cup 60. As shown in FIG. 22, each spheroidal cup 60 is adapted and oriented to mate with a respective first and second spheroidal dome 62. In vivo, each elastic element 64 is in sufficient compression to urge its respective spheroidal cup 60 onto its respective spheroidal dome 62. Thus, compression of the elastic elements 64, together with the musculature and supporting structures of the spine, will maintain the integrity of the prosthesis 10.

Exemplary materials for the spheroidal cup 60 and the spheroidal dome 62 include metal on plastic (MoP) (e.g., polyethylene, Ultra Highly Cross-Linked Polyethylene (UHXLPE), or Ultra High Molecular Weight Polyethylene (UHMWPE)); metal on metal (MoM) (e.g., cobalt-chromium alloy, titanium alloy, stainless steel, tantalum, zirconium); ceramic on plastic (CoP) (e.g., Ultra High Molecular Weight Polyethylene (UHMWPE)); and ceramic on ceramic (CoC).

Exemplary materials for the elastic element 64 such as a helical spring include a suitable biocompatible metal, including, but not limited to, stainless steel, cobalt-chromium alloy, and titanium and its alloys, including, but not limited to nickel-titanium alloy (nitinol).

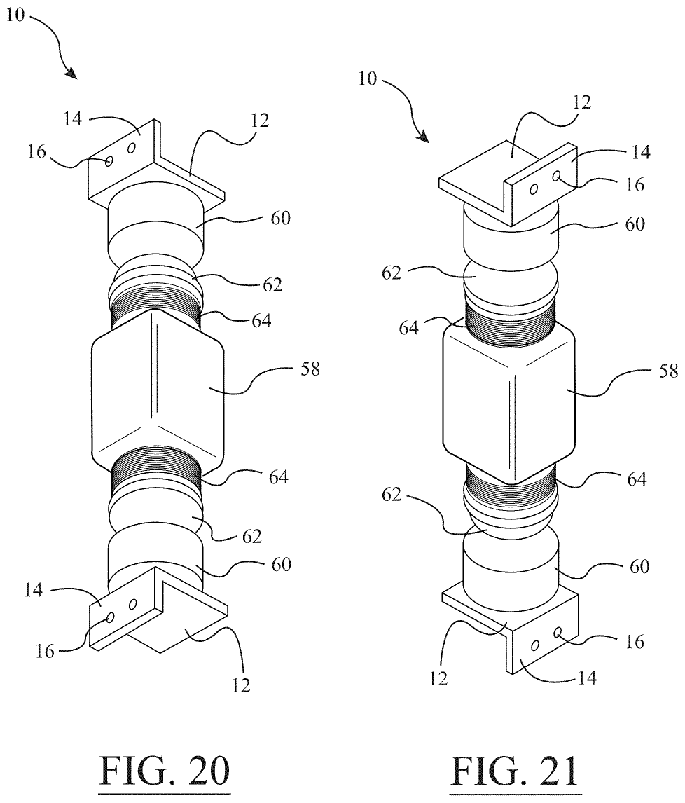

Turning now to FIGS. 20 and 21, exploded views of the prosthesis 10 show alignment of the various elements. A vertebral spacer 58 may be generally central in an axis to the prosthesis 10 and may be sized to cooperate with other elements of the prosthesis 10 to suitably occupy the gap created when a vertebra 70 (e.g., C5) and its adjacent discs 84 are removed. Operatively connected to the spacer 58 a first and second helical spring 14 is a first and second spheroidal dome 18. The spinal prosthesis 10 further comprises first and second anchors 12, each first and second anchor 12 adapted to be secured to a vertebra (e.g., C4 and C6). Operatively attached to each anchor 12 is a spheroidal cup 60. As shown in FIGS. 20 and 21, each spheroidal cup 60 is adapted and oriented to mate with a respective spheroidal dome 62. In vivo, each helical spring 64 is in sufficient compression and urges its respective spheroidal cup 60 onto its respective spheroidal dome 62. Thus, compression of the springs 64, together with the musculature and supporting structures of the spine, will maintain the integrity of the prosthesis 10.

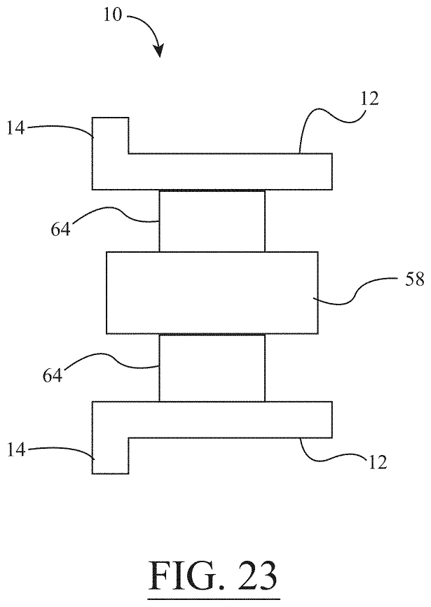

FIG. 22 illustrates a prosthesis 10 comprising, generally, a spacer 58 and anchors 12. The prosthesis 10 further comprises a sleeve 66 enclosing each articulating ball-and-socket-like joint combination (e.g., spheroidal dome 62, spheroidal cup 60, and elastic elements (e.g., springs 64 (FIGS. 19-21) and elastic columns (FIG. 23)). The sleeve 64 protects the joints and elastic columns from bodily fluids that could otherwise cause damage, allows for a packing around the joints and elastic columns to protect as well as lubricate, and encloses the expanding and collapsing structures so as to protect from entanglement with surrounding tissues. Finally, the sleeve prevents excessive contact with the tissues in order to reduce inflammatory reactions that could damage the surrounding structures.

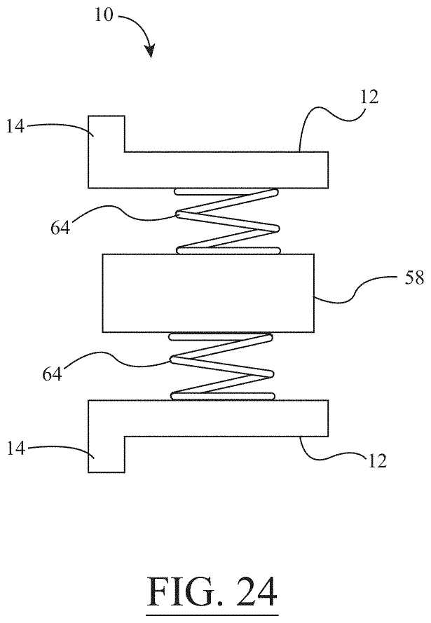

FIGS. 23 and 24 each illustrate prostheses 10 comprising a pair of elastic elements alone 64 with no articulating ball-and-socket-like joint combination (e.g., FIGS. 19, 22, and 23). The elastic elements 64, when elastic in compression, tension, and torsion, can mimic the normal elasticity of the spinal column and discs to allow sufficient resistance to such forces so as to provide stability and to avoid tissue injury or nerve or spinal cord damage yet provide sufficient elasticity so as to provide flexibility and allow for freedom of movement.

Exemplary materials for the elastic column 54 include, without limitation, elastic polyurethanes, elastomers, ChronoPrene™ (AdvanSource Biomaterials, Wilmington, Mass.), polylactic acid, and poly(3-hydroxybutyrate-co-3-hydroxyvalerate) (PHBV).

Making and sizing a prosthesis 10 requires, generally, determining the dimensions of the vertebrae above and below the level to be replaced. These dimensions can then be used to create the anchors 12 (e.g., 3-D printing or CAD development). The vertebral spacer 58 can be custom made utilizing CT scan (computerized tomography) or Mill (magnetic resonance imaging) to determine exact measurements. Or, a kit of standardized measurements may be employed with multiple options readily available in the operating room as is already common in most other medical devices. The elastic elements (e.g., 64) may be fixed, as the case may be, to the spheroidal dome 62 or spheroidal cup 60 or to the spacer 58 or anchor 12 by welding, adhesive, or other biocompatible mode of fixation. The entire prosthesis 10 should be at least the same height, but possibly taller than the material being removed, including the native vertebral body and the disc above and below.

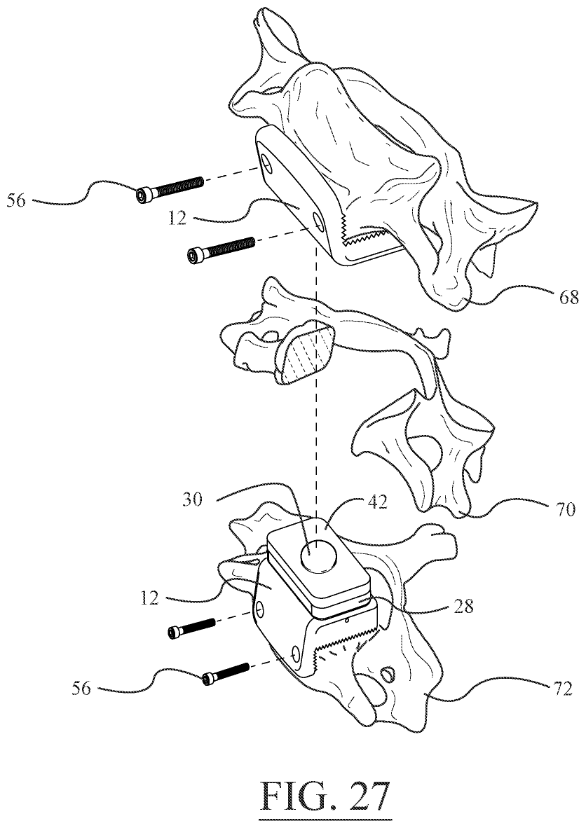

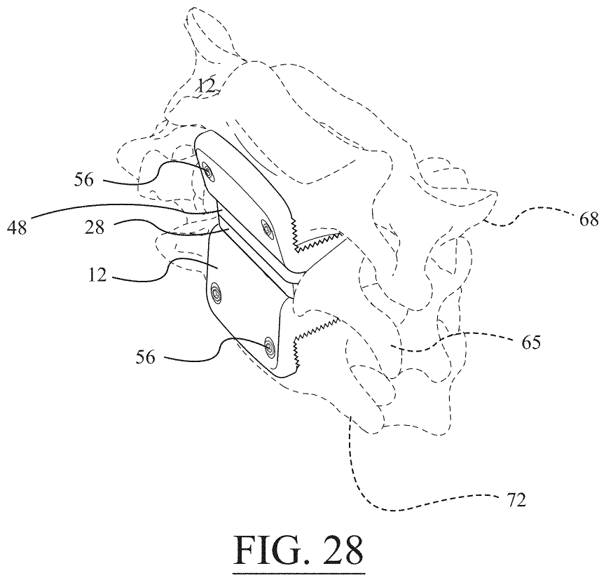

Finally, turning to FIGS. 25-28, the anatomical environment with the prosthesis 10 is shown. FIG. 25 shows an exemplary C4-C6 68, 70, 72 arrangement with accompanying discs 84. FIG. 26 shows C4-C6 68, 70, 72 expanded and modified to accept the prosthesis 10. The prepared surfaces 80, 86 are ready to accept the anchors 12 and the vertebral body of C5 has been removed 86. FIG. 27 is an expanded view of the prosthesis 10 in place and FIG. 28 shows the final arrangement with the prosthesis 10 in place.

To implant the prosthesis 10, the patient is placed on an operating table in supine position with the neck slightly extended. X-ray is used to confirm the appropriate spinal level. A transverse incision is made in the neck and the soft tissue is dissected until the platysma is encountered. The platysma is incised and blunt dissection employed to locate the avascular plane to the sternocleidomastoid. The muscle is swept laterally and the carotid sheath identified. The carotid sheath is retracted laterally and the trachea and esophagus identified medially. Blunt dissection is employed to expose the longus coli muscles. Electrocautery is used to dissect the muscles off the vertebral body. The anterior longitudinal ligament is next dissected with the electrocautery and the vertebral body and intervertebral discs are identified. A needle is inserted into the disc space and X-ray used to confirm correct spinal level. Next, Caspar pins are inserted into the C4 and C6 vertebral bodies. A spacer is attached and used to distract the vertebrae. A drill is next used to remove all of the vertebral body of C5 using the uncus as the lateral border for removal in order to preserve the vertebral arteries. The intervertebral discs are also drilled away and removed. The posterior longitudinal ligament is next identified, and depending on surgeon preference, incised and lifted away to reveal the dura of the spinal cord. Next, a measuring device is hammered into the space left from the removal of the C5 vertebral body. Ideally, the measuring device should fit snugly into the space and err on the side of being too large rather than too small. Once the size of the cavity is confirmed, the device may be assembled.

A kit of multiple sizes of each part is presented. The bottom anchor 12 with the ball component 28 injection molded into place is chosen based on correct sizing. The pad 42 is stretched and fit over top of the ball 30. The top anchor 12 is fitted snugly with the anchor socket 18 mating with the ball 30. An implanting device is affixed to the construct and used to place the device into the space. The endplates of the cervical vertebrae are next shaved with the drill to remove cortical bone to promote bone growth and fusion to the anchors 12 of the prosthesis 10. The prosthesis 10 is next hammered into place and positioning is confirmed with X-ray. The prosthesis 10 is secured to the vertebral bodies with screws, the Caspar pins are removed, and hemostasis is achieved. The wound is closed in multi-layered fashion.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

We claim:

1. (canceled)

2. A spinal prosthesis comprising:
a first anchor comprising:
a first anchor body comprising a groove; and
a first anchor leg formed at an angle to the first anchor body;
a second anchor comprising:
a second anchor body comprising a socket; and
a second anchor leg formed at an angle to the second anchor body;
a ball component comprising:
a base comprising a skirt extending therefrom, the skirt mated with the groove; and
a ball extending from the base, the ball mated with the socket, the ball component formed of medical grade silicone; and
a compressible elastic pad interposed between the ball component base and the second anchor, the pad comprising a hole therethrough, the pad elastically securing at least a portion of the ball in the hole.

3. A spinal prosthesis comprising:
a first anchor comprising a first anchor body;
a second anchor comprising a second anchor body and comprising a socket;
a ball component comprising:
a base adapted to connect to the first anchor body; and
a ball extending from the base, the ball adapted to mate with the socket; and
a pad comprising a hole therethrough, the pad adapted to secure at least a portion of the ball in the hole.

4. The spinal prosthesis of claim 3, wherein:
the ball component base is connected to the first anchor body;
the ball is mated with the socket; and
the pad is interposed between the ball component base and the second anchor, the pad securing at least a portion of the ball in the hole.

5. The spinal prosthesis of claim 3, wherein:
the first anchor further comprises a first anchor leg formed at an angle to the first anchor body; and
the second anchor further comprises a second anchor leg formed at an angle to the second anchor body.

6. The prosthesis of claim 4, wherein the ball component is connected to the first anchor body with a skirt-and-groove fitting.

7. The prosthesis of claim 6, wherein the ball component is secured to the first anchor body with a silicone adhesive.

8. The prosthesis of claim 6, wherein:
the first anchor body comprises at least one first anchor body duct extending from an outside surface of the first anchor body to the skirt-and-groove fitting, the first anchor body duct at least partially filled with the silicone adhesive; and
the skirt-and-groove fitting is at least partially filled with the silicone adhesive.

9. The prosthesis of claim 6, wherein:
the ball component base comprises at least one ball component base duct extending from an outside surface of the ball component base to the skirt-and groove fitting, the ball component base duct at least partially filled with a silicone adhesive; and
the skirt-and-groove fitting is at least partially filled with a silicone adhesive.

10. The prosthesis of claim 3, wherein the pad is compressible and elastic.

11. A spinal prosthesis comprising:
a first anchor adapted to be affixed to a vertebra and comprising a ball adapted to mate with a socket;
a second anchor adapted to be affixed to a vertebra and comprising a socket adapted to mate with the ball; and
a pad comprising a hole therethrough, the pad adapted to secure at least a portion of the ball in the hole.

12. The spinal prosthesis of claim 11, wherein:
the socket is mated with the ball; and
the pad is interposed between the first anchor and the second anchor, the pad securing at least a portion of the ball in the hole.

13. A kit of component parts for assembling a spinal prothesis for replacement of at least a portion of the body of at least one subject vertebra and adjacent discs, the at least one subject vertebra having first and second adjacent vertebrae, the kit comprising:
a first anchor adapted to be affixed to the first adjacent vertebra and comprising the groove of a skirt-and groove fitting;
a ball component comprising:
a base comprising the skirt of a skirt-and-groove fitting extending therefrom, the skirt adapted to mate with the first anchor groove; and
a ball extending from the base, the ball adapted to mate with a socket;
a compressible elastic pad comprising a hole therethrough, the pad adapted to elastically secure a portion of the ball in the hole; and
a second anchor adapted to be affixed to the second adjacent vertebra and comprising a socket, the socket adapted to mate with the ball.

14. The kit of component parts of claim 13, wherein:
the first anchor further comprises a first anchor duct extending from an outside surface of the first anchor to the groove; and
the ball component further comprises a skirt duct extending through the skirt and the skirt duct positioned to be in communication with the first anchor duct.

15. The kit of component parts of claim 13, wherein:
the skirt comprises a tab stop extending therefrom; and
the groove comprises a tab stop notch, wherein the tab stop is adapted to ride in the tab stop notch.

16. The kit of component parts of claim 13, wherein:
the ball component base comprises an arch stop extending therefrom;
the second anchor further comprises an arch stop groove; and
the pad further comprises a pad cut adapted to accept the arch stop, wherein the arch stop is adapted to ride in the pad cut and adapted to ride in the arch stop groove.

17. A kit of component parts for assembling a spinal prosthesis for replacement of at least a portion of the body of at least one subject vertebra and adjacent discs, the at least one subject vertebra having first and second adjacent vertebrae, the kit comprising:
a first anchor adapted to be affixed to the first adjacent vertebra, the first anchor comprising:
a body; and
a ball extending from the body, the ball adapted to mate with a socket;
a compressible elastic pad, the pad comprising a hole therethrough, the pad adapted to elastically secure a portion of the ball in the hole; and
a second anchor adapted to be affixed to the second adjacent vertebra, the second anchor comprising a body, the body comprising a socket, the socket adapted to mate with the ball.

18. A method of implanting a spinal prosthesis for replacement of at least a portion of the body of at least one subject vertebra and adjacent discs, the at least one subject vertebra having first and second adjacent vertebra, the prosthesis having:
a first anchor adapted to be affixed to the first adjacent vertebra and comprising a ball, the ball adapted to mate with a socket;
a compressible elastic pad comprising a hole therethrough, the pad adapted to elastically secure a portion of the ball in the hole; and
a second anchor adapted to be affixed to the second adjacent vertebra and comprising a socket, the socket adapted to mate with the ball, the steps comprising:
(a) surgically exposing the subject vertebra and removing the body of the subject vertebra;
(b) removing the adjacent discs;
(c) preparing the first adjacent vertebra to accept the first anchor;
(d) preparing the second adjacent vertebra to accept the second anchor;
(e) placing the prosthesis at least partially into the space created by the removal of the body of the subject vertebra;
(f) affixing the first anchor to the first adjacent vertebra; and
(g) affixing the second anchor to the second adjacent vertebra.

19. A method of implanting a spinal prosthesis for replacement of at least a portion of the body of at least one subject vertebra and adjacent discs, the at least one subject vertebra having first and second adjacent vertebra, the prosthesis having:
a first anchor adapted to be affixed to the first adjacent vertebra;
a ball component comprising:
a base connected to the first anchor; and
a ball extending from the base, the ball adapted to mate with the socket of a ball-and-socket fitting;
a compressible elastic pad comprising a hole therethrough, the pad elastically securing a portion of the ball in the hole; and
a second anchor adapted to be affixed to the second adjacent vertebra and comprising the socket of a ball-and-socket fitting, the socket mated with the ball, the steps comprising:
(a) surgically exposing the subject vertebra and removing the body of the subject vertebra;
(b) removing the adjacent discs;
(c) preparing the first adjacent vertebra to accept the first anchor;
(d) preparing the second adjacent vertebra to accept the second anchor;
(e) placing the prosthesis at least partially into the space created by the removal of the body of the subject vertebra;
(f) affixing the first anchor to the first adjacent vertebra; and
(g) affixing the second anchor to the second adjacent vertebra.
